(19) United States
(12) Patent Application Publication
(10) Pub. No.: US 2024/0082269 A1
(43) Pub. Date: Mar. 14, 2024

(54) METHODS AND PRODUCTS INCLUDING SPECIFIC CANNABINOIDS

(71) Applicant: Gregory L. Smith, St. Pete Beach, FL (US)

(72) Inventor: Gregory L. Smith, St. Pete Beach, FL (US)

(21) Appl. No.: 18/303,196

(22) Filed: Apr. 19, 2023

Related U.S. Application Data
(60) Provisional application No. 63/363,257, filed on Apr. 20, 2022.

(51) Int. Cl. A61K 31/00; A61K 9/00; A61K 31/05

(52) U.S. Cl. CPC A61K 31/658; A61K 9/006; A61K 31/05

(57) ABSTRACT

Products and methods are provided that use specific cannabinoid molecules to achieve various medical effects in human subjects. The varin includes tetrahydrocannabivarin (THCV), cannabidivarin (CBDV), and blends thereof. Varin may be used, in combination with specific secondary ingredients and via specific delivery mechanism, to achieve hair growth, prevent hair loss, treat metabolic disorders, or achieve weight loss.

### CROSS-REFERENCE TO RELATED APPLICATION

This application claims the benefit of U.S. Provisional Application No. 63/363,257, entitled “Methods and Products Including Specific Cannabinoids”, filed Apr. 20, 2022, the disclosure of which is hereby incorporated by reference in its entirety.

### TECHNICAL FIELD

The disclosure is directed to products that include specific cannabinoid molecules, and related methods of using said products for treatment of disorders in the human body.

### BACKGROUND

Cannabinoid molecules are a family of chemical compounds derived from the cannabis plant. At least 113 distinct cannabinoids have been isolated from cannabis. The most well-known cannabinoid may be phytocannabinoid tetrahydrocannabinol (THC), the primary psychoactive compound in cannabis. However, a number of other cannabinoid molecules have been identified and studied for their various uses. For example, cannabidiol (CBD) is a class of phytocannabinoid molecules that have been approved by the United State Food and Drug Administration for the treatment of epilepsy disorders.

Humans may suffer from disorders that adversely affect the lives of people. Hair loss is one of the most common syndromes affecting the lives of people. In particular, androgenic alopecia, also known as AGA, is very common in both men and women. Although androgenic alopecia may affect people of any age, the prevalence of androgenic alopecia increases with age. Also, the tendency of suffering from androgenic alopecia is different across the races, and more likely is found in men.

Metabolic disorders include weight gain, abdominal growth, abnormal blood sugar levels, and abnormal blood lipid ratios. Metabolic disorders or syndromes are increasingly prevalent in developed economies, with up to one-third of adults in the United States being affected by one or more metabolic disorders.

Accordingly, there is a need in the art for compositions and methods that include other cannabinoids that may be used to treat disorders in the human body such as hair loss and metabolic disorders.

### SUMMARY

In one aspect, the present disclosure is directed to a method for promoting hair growth in an affected area of a subject in need of such treatment, the method comprising: applying a composition comprising a hair growth-effective amount of a varin to the affected area topically for a time sufficient to promote hair growth.

In another aspect, the present disclosure is directed to a method for preventing hair loss in an affected area of a human subject in need of such treatment, the method comprising: applying a composition comprising an effective amount of a varin to the affected area topically for a time sufficient to promote hair growth.

In another aspect, the present disclosure is directed a method for eliciting a health-improving metabolic effect in a human subject, the health-improving metabolic effect being selected from the group consisting of weight loss, reducing A1c, lipids, abdominal girth, and combinations thereof, the method comprising: administering to the human subject a mucoadhesive polymer impregnated with a varin; the varin being present in an amount of between 1 mg and 16 mg per daily dose.

Other systems, methods, features, and advantages of the disclosure will be, or will become, apparent to one of ordinary skill in the art upon examination of the following detailed description. It is intended that all such additional systems, methods, features, and advantages be included within this description and this summary, be within the scope of the disclosure, and be protected by the following claims.

### DETAILED DESCRIPTION

Broadly, this disclosure provides compositions and methods including cannabinoids that are varins. A varin is a cannabinoid homologue having two fewer carbon atoms than the corresponding cannabinoid. Most classical cannabinoids are 21-carbon compounds. However, some cannabinoids include fewer than 21 carbon atoms, primarily because of variation in the length of the side-chain attached to the aromatic ring. For example, in THC, CBD, and CBN, this side-chain is a pentyl (5-carbon) chain. In the most common varin homologue, the pentyl chain is replaced with a propyl (3-carbon) chain. Cannabinoids with the propyl side chain are named using the suffix varin and are designated THCV, CBDV, or CBNV.

Generally, a varin will have a reduced (or nonexistent) psychoactive effect compared to the corresponding cannabinoid. Namely, THCV will not have significant psychoactive effects as THC does.

THCV has CAS number 31262-37-0. CBDV has CAS number 24274-48-4.

In embodiments of the disclosure, a varin is selected from the group consisting of tetrahydrocannabivarin (THCV), cannabidivarin (CBDV), and blends thereof.

In embodiments of the disclosure, these varins offer advantageous medical properties at particular dosages, and further in conjunction with specific secondary ingredients.

Hair Loss and Regrowth

Hair growth, and subsequent shedding or loss of hair, occurs in a cycle composed of four distinct phases. The first three phases are the anagen, catagen, and telogen phases. These phases cover the growth and maturation of hair and the activity of the hair follicles that produce individual hairs. During the fourth, or exogen, phase, aged hair falls out naturally, or is shed. Typically, a new hair is developing to replace the lost hair.

The anagen phase is the longest of the four phases. The anagen phase lasts about 3 years to 5 years, but may extend to about 7 years. This phase differs with different types of hair. For example, the anagen phase for eyebrow hairs and other body hair, is much shorter than the anagen phase for scalp hair. During the anagen phase, hair follicles push out hairs that continue to grow until they reach the end of their lifespan and fall out naturally. As much as about 80 percent to 90 percent of the hairs on the head are in the anagen phase.

The catagen phase starts when the anagen phase ends. The catagen phase lasts about 10 days. The hair follicle shrinks and hair growth slows during the catagen phase. The hair also separates from the bottom of the hair follicle, yet remains in place during its final days of growth. About 5 percent of the hairs on the head are in the catagen phase.

The telogen phase typically lasts about 3 months. About 10 to about 15 percent of scalp hairs are in this phase. Hairs do not grow during the telogen phase, but typically they do not fall out either. A new hair starts to form in follicles that have just released a hair during the catagen phase.

The exogen phase typically is considered an extension of the telogen stage. During the exogen phase, hair is shed. Hair washing and brushing facilitates loss, as does friction with clothing. Loss of between about 50 and about 100 hairs per day is not uncommon. During this exogen phase, new hair grows in the follicle from which an old hair has fallen away or has been removed. The exogen phase is as short as about 2 months and about 5 months.

Male pattern baldness is the male pattern phenotype of androgenic alopecia. The Norwood scale may be used to characterize male pattern baldness. The scale identifies 7 stages that measure severity and pattern of hair loss.

Stress also may cause premature hair loss. For example, trichotillomania is a psychological inability to resist pulling hair from the body. This syndrome is treatable with psychoactive drugs, such as anti-depressants. Androgenic alopecia may be treated in accordance with a method of the disclosure. This and other syndromes, such as non-scarring alopecia, also may be treated in accordance with a method of the disclosure.

In one aspect, the disclosure is directed to a method for promoting hair growth in an affected area of a subject in need of such treatment. In another aspect, the disclosure relates to a product for promoting hair growth in an affected area of a subject in need of such treatment. In another aspect, the disclosure is related to a method and product for reducing hair loss in an affected area of a subject in need of such treatment.

In accordance with embodiments of the disclosure, the method for promoting hair growth comprises topically applying a composition comprising a hair growth-effective amount of a varin to an affected area of a subject in need of such treatment. The composition is applied for a time sufficient to promote hair growth. In embodiments of the disclosure, a composition is disclosed that comprises a hair growth-effective amount of a varin.

In accordance with other embodiments of the disclosure, the method for reducing hair loss comprises topically applying a composition comprising a hair-loss reducing amount of a varin to an affected area of a subject in need of such treatment. The composition is applied for a time sufficient to reduce hair loss. In embodiments of the disclosure, a composition is disclosed that comprises a hair loss-reducing amount of a varin.

In various embodiments of this disclosure, the time sufficient to promote hair growth or the time sufficient to reduce hair loss may be several days, or several weeks, or several months.

In embodiments of the disclosure, the amount of varin also may be called a therapeutically effective dose or dosage.

Any area of hair growth may be treated in accordance with embodiments of the disclosure. Methods and compositions for hair growth are applicable to the human scalp, eyelashes, and eyebrows. Similarly, any area of hair loss may be treated in accordance with embodiments of the disclosure.

In accordance with embodiments of the disclosure, a composition including one or more cannabinoid varins may be applied topically to a human body at least once per day. The composition may be applied at least once daily for a period of 50 to 120 days in order to achieve hair regrowth. In some embodiments, continued topical usage of a composition in accordance with this disclosure may be life-long in order to prevent reoccurrence of the hair loss related condition.

In embodiments of the disclosure directed to hair growth, the composition may include a total amount of varins of between 3 mg and 12 mg per dose. In some embodiments, the total amount of varins may be less than 12 mg per dose; or less than 10 mg per dose, or less than 8 mg per dose. In other embodiments, the total amount of varins may be greater than 1 mg per dose; or greater than 3 mg per dose; or greater than 5 mg per dose. In yet other embodiments, the total amount of varins may be between 1 mg and 12 mg per dose; or between 3 mg and 10 mg per dose; or between 5 mg and 8 mg per dose. Generally, these dosages may be applied once per day. However, a twice daily dosage having half of the amounts listed above would also result in the same total daily dosage.

Of the total varins, the composition may include THCV, CBDV, and combinations thereof. In some embodiments of the disclosure, the composition may include both THCV and CBDV. In such embodiments, of the total varins, the composition may include between 1 mg and 4 mg of THCV and between 2 mg and 8 mg of CBDV per dose.

These dosage amounts may be considered as the hair growth effective amounts of the varins, that are delivered to the human body to promote hair growth thereon. These dosage levels may be advantageous to achieving hair regrowth, as incorrect dosing may lead to hair loss. For example, significant overdosing may reverse the desired therapeutic effects.

CBD is a partial CB1 (cannabinoid receptor type 1) blocker. Varins in embodiments of the disclosure are full CB1 blockers. Blocking CB1 receptors in hair follicles in animals and humans has been shown to stimulate hair shaft elongation via keratinocytes, and thickness (volume) of hair shaft via stimulation of matrix cells, essentially leading to onset (or elongation) of the anagen phase. Additionally, varins may also prevent hair loss by pushing more of the hair follicles into the anagen phase.

In embodiments of the disclosure, the composition may further include secondary active ingredients that may further help achieve hair growth in a human subject. Namely, the composition may include menthol as a secondary active ingredient. As is generally known, menthol is a naturally occurring and synthetically available organic compound having CAS number 2216-51-5 or 89-78-1. Menthol used in the compositions of this disclosure may either be naturally occurring peppermint extract or synthetic pure menthol, or combinations thereof.

Menthol has been shown in animals to be a blocker of Fibroblast growth factor 5 (FGF5). In embodiments of the disclosure, blocking FGF5 may result in prolonged anagen (growth) phase of the hair follicle and increased hair growth. Menthol may synergistically further enhance the hair growth effect by the varins as discussed above.

The dosage of menthol in embodiments of methods of the disclosure of treating hair loss may be about 75 mg to about 225 mg of menthol delivered to the human body per day. In other embodiments, the dosage of menthol may be between about 100 mg and about 200 mg per day; or between about 125 mg and about 175 mg per day; or about 150 mg per day. The weight percentage of menthol in a composition in accordance with this disclosure may be about 3% by weight, or from about 1% to about 5% by weight.

The composition may further include non-active ingredients.

Non-active ingredients in the composition used for hair growth may include ethanol, emu oil, HFA 134A (1,1,1,2-tetrafluoroethane) propellant, and dimethicone. In embodiments, a composition of the disclosure may include approximately 5 grams of ethanol, six hundred grams of Emu oil, 14.9 grams of HFA 134A (1,1,1,2-tetrafluoroethane) propellant, and nine hundred milligrams of dimethicone. In embodiments of the disclosure, the amounts of the non-active ingredients may be based on the desired consistency of the composition to be applied to the scalp.

In embodiments of the disclosure, a composition for hair growth having an effect on the body different from the effect of a varin may be used in combination with a varin. In embodiments of the disclosure, minoxidil, finasteride, and other compositions may be applied in conjunction with a varin to promote hair growth.

Methods in accordance with embodiments of the disclosure may also further include administering ingested compositions that include varins, in combination with topical application of compositions discussed above, to cause hair growth in a human subject. Ingested compositions may be in the form of an oral spray or a muco-adhesive strip that delivers one or more active ingredients.

In embodiments in accordance with the disclosure, a method for causing hair growth may include a combination of a topical treatment and an ingested oral treatment. The ingested oral treatment may be in the form of a once daily oral hemp-derived extract, which will be used as an adjunct to the topical application. The topical formulation is expected to impact the hair follicle receptors in the immediate area around the point of application of the topical. The oral formulation will be absorbed directed from the intra-oral mucosa into the blood stream. The oral formulation may contain varins and CBD that will have a systemic effect on all hair follicles, not just those in the area of topical application. By providing the varins and CBD through systemic absorption, rather than only topical route, hair follicle maintenance may be increased in the anagen phase for all hair follicles on the scalp, eyebrows, and eyelashes.

The oral product may contain CBD in an amount of between about 25 mg and about 50 mg per daily dose, and varins (CBDV and THCV) combined in an amount of about 10 mg per daily dose.

In some embodiments of the disclosure, the composition administered orally may be a mouth strip composed of a mucoadhesive material impregnated with L-Arginine and menthol in addition to the hair growth modifying composition. Studies over the past decade have shown that menthol increases intraoral penetration, also known as flux, of several medications as well as other compounds such as nicotine. L-Arginine applied locally has been shown to enhance vasodilation within the mucosa. Once absorbed, L-Arginine is converted to nitric oxide (NO), known as endothelium-derived relaxation factor (EDRF), which causes vasodilatation of the mucosa.

Mucoadhesive delivery has been developed to enable more prolonged retention at the site of application. Mucoadhesive polymers have numerous hydrophilic groups, such as hydroxyl, carboxyl, amide, and sulfate. These groups attach to mucus membranes by various interactions such as hydrogen bonding and hydrophobic or electrostatic interactions. These hydrophilic groups also cause polymers to swell in water and, thus, expose the maximum number of adhesive sites. The extended retention time results in enhanced bioavailability and maximum drug concentration in the plasma (Cmax), and decreased time until maximum concentration in the plasma (Tmax) of the active ingredient of the varins. The mucoadhesive polymer may be placed on any mucosal area. In embodiments of the disclosure, the mucoadhesive polymer is adhered to a buccal or sublingual surface.

Metabolic Disorders and Weight Loss

This disclosure also provides a method for eliciting a health-improving metabolic effect selected from the group consisting of reducing A1c, lipids, abdominal girth, and combinations thereof, in a subject in need of such an effect, the method comprising: administering to the subject a composition comprising a mucoadhesive polymer impregnated with an effective amount of a varin. Broadly, methods in accordance with this disclosure may aid in treating metabolic disorders by causing weight loss through the administration of a mucoadhesive including a varin.

In embodiments of the disclosure, the varin used in the method for causing a health-improving metabolic effect may be selected from the group consisting of tetrahydrocannabivarin, cannabidivarin, and blends thereof. In embodiments of the disclosure, the varin may be tetrahydrocannabivarin (THCV) specifically. In other embodiments of the disclosure, the varin may be cannabidivarin (CBDV) specifically.

In some embodiments of the disclosure, the dosage of active ingredient in the method is from 1 mg to 16 mg of THCV per daily dose, and from 1 mg to 16 mg of CBDV, or any combination of the two. Generally, the total amount of varins should be between 1 mg to 16 mg per daily dose. In some embodiments, the dosage of active ingredient may be from 8 mg to 16 mg per once daily dose.

This disclosure also provides the mucoadhesive product infused with the varin.

The use of the mucoadhesive strip (which contains the active ingredients) increases the intraoral blood flow and increases the flux between the active hemp-oil ingredient and the mucous membranes, as compared to other absorption rates such as ingestion. Generally, a mucoadhesive strip in accordance with this disclosure is placed into a user's mouth and allowed to dissolve therein. Oromucosal drug delivery is highly attractive due to the ease of administration without the need of swallowing, and improved patient safety. Further, the mucoadhesive strip has been proven in human studies to result in increased intraoral absorption of the active ingredients directly into the bloodstream compared to swallowing and digesting the hemp-oil.

Generally, the mucoadhesive strip helps the hemp-oil bypass the catabolic effects of the liver and gastrointestinal system, thereby achieving an improved delivery mechanism for the active ingredient varin.

In some embodiments, the product has tetrahydrocannabivarin (THCV) as an active ingredient. THCV is a neutral CB1 blocker (antagonist). Blocking the CB1 receptor has been shown in humans and animals to have effects on the hypothalamus (brain) resulting in decreased appetite, decreased food seeking behavior and earlier onset of satiation. Blocking the CB1 receptor in the gut results in improved insulin-sensitivity, and lipolysis (breakdown) of ventral fat.

In other embodiments of the disclosure, CBDV may be an active ingredient.

The mucoadhesive strip may further include other non-active ingredients, such as menthol or peppermint. In one embodiment, menthol may be present in the mucoadhesive strip in an amount of about 1.5% by weight.

### Comparative Examples—Effectiveness of CBD, THCV, and CBDV

Four studies have been conducted that confirm statistically significant weight loss, decrease of abdominal girth, and decrease in A1C blood levels (diabetic measure) with daily use of 8 mg doses of THCV over 60-180 days.

The following pre-clinical and clinical research has shown that small daily oral amounts of Hempson Oil®—a hemp-extract high in cannabidiol (CBD), tetrahydrocannabivarin (THCV), and cannabidivarin (CBDV)—have consistent effects of weight loss, reduction in abdominal girth, blood sugar (HgbA1c) and blood lipid ratios. These comparative examples are shown merely for the effect the active ingredients may have upon metabolic disorders and weight loss. However, these comparative examples do not use mucoadhesive strips.

These studies show that the primary active ingredient for weight loss and metabolic benefits was due to the THCV component. THCV acts by neutrally blocking the cannabinoid one (CB1) receptor in the hypothalamus (brain) and in the gut (pancreas, liver, and intestinal tract). CB1 blockade switches the body into the ‘fasting’ state so that the body preferentially uses stored fat in the abdomen.

By blocking the CB1 receptor, the hypothalamus orchestrates various activities in the brain and body so that the fat around our organs (ventral fat) is preferentially used up, the insulin receptors on muscles become more sensitive resulting in decreased blood sugars, decreased insulin excretion, and our liver modifies the ratio of good to bad cholesterol (LDL/HDL) and we stop producing ‘hunger pangs.’ CBD is a partial CB1 blocker, whereas THCV and CBDV are both full (complete) CB1 blockers.

The Hempson Oil Studies: from 2019 until April 2021, several independent high quality human trials were completed using an oral Hempson® oil in a micellized tincture or non-micellized oil in gel caps. All of the studies were NIH-supported. All studies were double-blind, placebo-controlled, multisite, and randomized effectiveness studies of a hemp extract, known as Hempson® oil that is high in CBD, THCV, and CBDV. The studies used either type II diabetics, obese or healthy adults. The studies done with micellized oil tincture used lower doses with micellized Hempson® oil because of increased the absorption and bioavailability of the cannabinoids. The research from the studies confirms the expected benefits from other human and animal studies of weight loss, decreased abdominal girth, significantly improved blood sugar levels, healthier blood lipid ratios, reduced blood pressure, as well as other statistically significant improvements in sleep onset and duration, mood, arthritic pain, and several inflammatory markers in the blood.

Initial screening of the subjects for inclusion into the study included: aspartate transaminase (AST), alanine aminotransferase (ALT), to assess liver function, creatinine and blood urea nitrogen (BUN), thyroid stimulating hormone (TSH) for the evaluation of the thyroid and to help evaluate kidney function, a complete blood count (CBC), and platelets were drawn. Each of these tests was run on arterial blood drawn following standardized protocol for the procedures and completed using standard techniques.

In each study, subjects were randomly assigned to either the Hempson Oil® group (100 subjects) or the control group (50 subjects). Each subject signed a study consent form. Subjects were instructed to orally ingest this study product once daily. All subjects were instructed not to change their eating, drinking, or exercise habits for the duration of this study.

Study 1 (2019)—150 healthy adults (50 placebo, 100 used 12 mg micellized Hempson®) for 90 days.

Study 2 (2019)—150 type II diabetics (50 placebo, 100 used 12 mg micellized Hempson®) for 180 days.

Study 3(2020)—150 healthy adults (50 placebo, 100 used 12 mg micellized Hempson®) for 90 days.

Study 4 (2021)—150 obese adults (50 placebo, 50 in Group 1 (50 mg Hempson®) 250-300 lbs, 50 in Group 2 (100 mg Hempson®) over 300 lbs) for 90 days.

In all studies the placebo group did not have any statistically significant changes between day 1 and the final day of the study. No safety issues or significant adverse reactions were documented during the course of any of these trials. All of the study results reported below were statistically significant (p<0.05) compared to placebo results.

Weight Loss results: Study 2—Weight (lbs)—before=186.5; after 180 days=172.8; difference=13.7 lbs or 8.7% weight loss. Study 4—Weight (lbs)—Group 1—9.2 lbs weight loss, Group 2—11.2 lbs weight loss, after 90 days.

Abdominal Girth results: Study 2—Inches of girth—before=44.81; after 180 days=41.08; difference=3.73 inches loss, after 180 days. Study 4—Inches of girth difference—Group 1—2.8 inches, Group 2—1.6 inches, both after 90 days.

Blood Sugar (HbgA1C) results: Study 1—HgbA1C—before=5.2; after 90 days=4.7; difference=11% decrease in diabetic marker after 90 days. Study 2—HgbA1C—before=6.84; after 180 days=5.67; difference=17% decrease in diabetic marker after 180 days.

Lipid Ratios (elevation of HDL) results: Study 1—HDL—before=56.62; after 90 days=61.27; difference=8.2% increase in ‘good cholesterol’.

Blood Pressure (BP) results: Study 2—Systolic BP—before=137.22; after 180 days=127.24; difference=10 mm Hg reduction in BP after 180 days.

Summary of comparative example results: Several placebo-controlled studies of Hempson® Oil over 90-180 days revealed several statistically significant and clinically significant benefits in weight loss, decreased abdominal girth, blood sugar, HDL cholesterol levels and blood pressure. These findings were consistent with previous pre-clinical studies in animal models, and with studies of similarly acting synthetic CB1 blockers.

### Examples—Study of THCV Impregnated Mucoadhesive Strip

A mucoadhesive polymer impregnated with THC was compared to an ingestible gummy having a substantially similar amount of THC. This study used THC as an active ingredient for the purpose of testing the effectiveness of the mucoadhesive mouth strip. This use of THC-containing mucoadhesive polymer is expected to be exemplary of use of THCV and other varins as active ingredients in a mucoadhesive strip.

Upon testing, the mucoadhesive mouth strip had superior pharmacologic performance compared to an ingested gummy. There was an earlier Tmax of 60-70 minutes for the mucoadhesive mouth strip compared to 90 minutes for the gummy. This is evidence of approximately 30% faster absorption of the THC into the blood stream in the mucoadhesive mouth strip group. The Cmax was statistically the same for both groups, meaning they both achieved the same level of THC in the blood stream. However, the mucoadhesive mouth strip group achieved a steady state in the blood stream by 45 minutes and maintained the steady level for twice as long as the gummy group. The gummy group did not show steady state. Instead, it peaked at 90 minutes followed by a rapid trough, which is less physiologically beneficial than a smooth steady level of THC in the blood seen with the mouth strip. The rapid decline in the THC level in the gummy group compared to the smoother decline in the moth strip group supports a shorter duration of effect of the gummy compared to the mucoadhesive mouth strip.

There was a very statistically significant difference in the metabolism of THC into the inactive (waste) metabolite Carboxy THC. The gummy group had several times the level of Carboxy THC at all times compared to the mucoadhesive mouth strip group. This is evidence of superior bioavailability of the mucoadhesive mouth strip THC compared to the gummy, as the THC from the gummy is being much more rapidly turned into inactive Carboxy THC. This type of study is limited in its ability to provide exact bioavailability percentages. In fact there are only a handful of available studies in the literature where actual bioavailability has been calculated due to the cost and complexity of the required measurements. However, looking at the area under the curves (AUC) it is reasonable to conclude that the mouth strip had approximately 30% greater bioavailability of THC than the gummy.

Review of the blood levels over time reveals that the mouth strip had a more steady and earlier pattern of absorption compared to the gummy which had a peak at 90 minutes then rapid decline. The mouth strip had superior sustained levels compared to the gummy. The mouth strip had approximately 30% greater bioavailability than the gummy. This would make the euphoric effects from the mouth strip occur earlier, be more potent, consistent and of a longer duration than the gummy.

It is expected that this THC-based example illustrates the bioactivity and concentration or availability of varins, including THCV, CBDV, and CBNV.

While various embodiments of the disclosure have been described, the description is intended to be exemplary, rather than limiting and it will be apparent to those of ordinary skill in the art that many more embodiments and implementations are possible that are within the scope of the disclosure. Accordingly, the disclosure is not to be restricted except in light of the attached claims and their equivalents. Also, various modifications and changes may be made within the scope of the attached claims.

We claim:

1. A method for promoting hair growth in an affected area of a subject in need of such treatment, the method comprising:
applying a composition comprising a hair growth-effective amount of a varin to the affected area topically for a time sufficient to promote hair growth.

2. The method of claim 1, wherein the varin includes tetrahydrocannabivarin or cannabidivarin.

3. The method of claim 1, wherein the compound further comprises a therapeutically effective dosage of menthol-6 of between about 75 mg and about 225 mg.

4. The method of claim 1, wherein the affected area includes the scalp, the eyelashes, and the eyebrows.

5. The method of claim 1, wherein the affected area has experienced hair loss as a result of androgenetic alopecia, stress-related alopecia, or other non-scarring alopecia.

6. The method of claim 1, wherein the hair growth-effective amount of varin is between about 3 mg and about 12 mg per application.

7. The method of claim 2, wherein the hair growth-effective amount of tetrahydrocannabivarin is between about 1 mg and about 4 mg per application.

8. The method of claim 2, wherein the hair growth-effective amount of cannabidivarin is between about 2 mg and about 8 mg per application.

9. The method of claim 2, wherein the hair growth-effective amount of tetrahydrocannabivarin is between about 1 mg and about 4 mg per application, and the hair growth-effective amount of cannabidivarin is between about 2 mg and about 8 mg per application.

10. The method of claim 1, wherein the method further comprises administering an ingested oral treatment in combination with the topical treatment;
the ingested oral treatment being in the form of a sublingual formulation including CBD in an amount of between about 25 mg and about 50 mg per daily dose, and varins comprising cannabidivarin and tetrahydrocannabivarin combined in an amount of about 10 mg per daily dose.

11. A method for preventing hair loss in an affected area of a human subject in need of such treatment, the method comprising:
applying a composition comprising an effective amount of a varin to the affected area topically for a time sufficient to promote hair growth.

12. The method for preventing hair loss of claim 11, wherein the varin includes tetrahydrocannabivarin or cannabidivarin.

13. The method of preventing hair loss of claim 12, wherein the effective amount of varin is between about 3 mg and about 12 mg per daily application.

14. A method for eliciting a health-improving metabolic effect in a human subject, the health-improving metabolic effect being selected from the group consisting of weight loss, reducing A1c, lipids, abdominal girth, and combinations thereof, the method comprising:
administering to the human subject a mucoadhesive polymer impregnated with a varin;
the varin being present in an amount of between 1 mg and 16 mg per daily dose.

15. The method of claim 14, wherein the varin is tetrahydrocannabivarin.

16. The method of claim 14, wherein the health-improving metabolic effect is weight loss.

17. The method of claim 14, wherein the varin is selected from the group consisting of tetrahydrocannabivarin, cannabidivarin, and blends thereof.

18. The method of claim 14, wherein the varin is present in an amount of between 8 mg and 16 mg per daily dose.

19. The method of claim 14, wherein the mucoadhesive polymer further includes menthol in an amount of about 1.5% by weight of the polymer.

20. The method of claim 14, wherein the method includes daily administration to the human subject of about 8 mg of tetrahydrocannabivarin per day, for a period of between 60 and 180 days.
